(19) United States
(12) Patent Application Publication
(10) Pub. No.: US 2025/0090307 A1
(43) Pub. Date: Mar. 20, 2025

(54) METHOD FOR SUTURING VALVE LEAFLET AND INTERVENTIONAL VALVE

(71) Applicant: KINGSTRONBIO (CHANGSHU) CO., LTD., Jiangsu (CN)

(72) Inventors: Shengping ZHONG, Jiangsu (CN); Yongfu JIN, Jiangsu (CN)

(21) Appl. No.: 18/550,734

(22) PCT Filed: Nov. 11, 2021
(86) PCT No.: PCT/CN2021/129999

§ 371 Date: Sep. 15, 2023

(30) Foreign Application Priority Data
Oct. 16, 2020 (CN) 202011111585.6

(51) Int. Cl. A61F 2/24

(52) U.S. Cl. CPC A61F 2/2415; A61F 2220/0075; A61F 2240/001

(57) ABSTRACT

A method for suturing a valve leaflet includes: demarcating first fixing edges (11) on both sides of a leaflet (1) and a second fixing edge (12) at bottom of the leaflet (1); folding the first fixing edges (11) towards a region between a valve skirt (2) and the leaflet (1), and suturing the first fixing edges (11) onto the valve skirt (2); and causing the second fixing edge (12) to be a freely stretching state, and suturing the second fixing edge (12) onto the valve skirt (2).

### CROSS-REFERENCE TO RELATED APPLICATIONS

The present application is U.S. National Stage Application of International Application No. PCT/CN2021/129999, filed on Nov. 11, 2021, which claims priority to Chinese Application No. 202011111585.6, filed on Oct. 16, 2020, the contents of both of which are incorporated herein by reference in their entirety.

### TECHNICAL FIELD

The present disclosure relates to the field of medical instrument technologies, and in particular, to a method for suturing a valve leaflet and an interventional valve.

### BACKGROUND

Biological valve prostheses open and close a leaflet through forward and backward flowing of blood. When the blood flows forward, the leaflet is opened to form a flow channel. When the blood flows backward, the leaflet is closed to prevent the blood from flowing. Both opening and closing require rapid action of the leaflet, and mal-operation or inaction are not allowed absolutely.

For a valve prosthesis, in natural state, a leaflet is in a closing state, which not only ensures that the leaflet can be opened smoothly when the blood flows forward, but also ensures that the leaflet can be closed quickly when the blood flows backward.

In natural state, the leaflet is in a closing state, and an edge of the leaflet connected to a valve skirt need to be folded and sutured towards the region between the valve skirt and the leaflet, so that various leaflets can close a pathway of the valve by using the tension of the leaflet after they are spliced. However, if the edge of the leaflet is sutured outward, various leaflets will separate from each other due to the tension of the leaflet. During the blood flows backward, the valve closure may be delayed, increasing regurgitation of the blood, or even resulting in major accidents in which a closing action cannot be created. Therefore, a scheme of suturing after folding the edge of the leaflet towards the region between the valve skirt and the leaflet is generally adopted in the related art.

Replacing a diseased valve in a human body by an interventional method is a new technology developed in recent years, in which a tailored valve is installed in a delivery system, and the valve is delivered by the delivery system to a site of the diseased valve through an arterial vessel or a venous vessel, and is replaced. Due to the limited size of human arterial and venous vessels, the diameter of the delivery system is limited.

When the interventional valve is installed in the delivery system, the interventional valve is compressed into a columnar structure, and no gap exists in the interventional valve. However, since the edge of the leaflet in the related art is sutured to the region between the valve skirt and the leaflet after being folded, the leaflet is in a form of double-layer at the bottom edge thereof, resulting in increasing the volume of the valve at the bottom of the leaflets after being compressed, which is not conductive to being loaded into the delivery system and implanted into the human body through the delivery system.

### SUMMARY

The present disclosure relates to a method for suturing the valve leaflet and an interventional valve in order to solve the following problems: the edge of the leaflet connected to the valve skirt is folded to a region between the valve skirt and the leaflet and then sutured, the valve after being compressed has an increased volume at the bottom position of the leaflet, which is not conductive to being inserted into the delivery system and implanted into the human body through the delivery system.

The object of the present disclosure is to provide a method for suturing a valve leaflet.

The method includes:
demarcating first fixing edges on both sides of a leaflet and a second fixing edge at a bottom of the leaflet;
folding the first fixing edges towards a region between a valve skirt and the leaflet, and suturing the first fixing edges onto the valve skirt; and
causing the second fixing edge to keep in a freely stretching state, and suturing the second fixing edge onto the valve skirt.

In an embodiment, after demarcating the first fixing edges on both sides of the leaflet and the second fixing edge at the bottom of the leaflet, the method further includes:

providing a transition hole position at a transition region that is formed between the first fixing edges and the second fixing edge.

In an embodiment, after providing the transition hole position, the method further includes:
cutting an incision at an edge of the transition region.

In an embodiment, a distance between one end of the incision away from the edge of the transition region and the transition hole position is ⅓ to ⅔ of a distance between the transition hole position and the edge of the transition region, and the incision extends in a direction from the edge of the transition region to the transition hole position.

In an embodiment, when a suture line passes through the valve skirt and the second fixing edge in sequence before entering the transition region, the method further includes:
after the suture line passes through the valve skirt and the second fixing edge in sequence, causing the suture line to pass through the transition hole position in a direction towards the valve skirt; and
causing the suture line to pass through the first fixing edges and the valve skirt in sequence.

In an embodiment, when a suture line passes through the second fixing edge and the valve skirt in sequence before entering the transition region, the method further includes:
after the suture line passes through the second fixing edge and the valve skirt in sequence, causing the suture line to pass through the valve skirt and the first fixing edges in sequence without passing through the transition hole position.

The present disclosure further provides an interventional valve manufactured by the method for suturing the valve leaflet provided in the present disclosure. The interventional valve includes a valve frame, the valve skirt and the leaflet. The valve skirt covers an inner wall surface of the valve frame. A first fixing edge and a second fixing edge of the leaflet are fixedly sutured on the valve skirt.

The technical solutions provided in the present disclosure can achieve the following advantageous effects.

According to the interventional valve and the method for suturing the valve leaflet, the first fixing edges on both sides of the leaflet are folded towards the region between the valve skirt and the leaflet, so that the leaflet can be smoothly opened when the blood flows forward when the leaflet is under tension, and the leaflet can be quickly closed when the blood flows backward; and the second fixing edge located at the bottom of the leaflet is not folded, so that the thickness of the bottom of the leaflet can be effectively reduced after suturing, thereby reducing the local volume of the interventional valve at the bottom of the leaflet after compression, and improving the insertion of the interventional valve into a delivery system and the implantation of the interventional valve into the human body.

It should be understood that both the foregoing general description and the following detailed description are just exemplary, but not restrictive to the present disclosure.

### BRIEF DESCRIPTION OF DRAWINGS

FIG. 1 is a flowchart of a method for suturing a valve leaflet according to an embodiment of the present disclosure;

FIG. 2 is a schematic diagram of a leaflet in a fully unfolding state;

FIG. 3 is a schematic diagram showing a state of the leaflet after suturing;

FIG. 4 is a schematic diagram showing states of the leaflet and the valve skirt after suturing;

FIG. 5 is a top view of the leaflet sutured on the valve skirt;

FIG. 6 is a schematic diagram showing a state of the edge of the leaflet after being folded; and

FIG. 7 is a schematic diagram of an edge of a leaflet in a freely stretching state.

### REFERENCE SIGNS

1—leaflet;
   11—first fixing edge;
   12—second fixing edge;
   13—transition hole position;
   14—incision;
   15—free edge;
   16—first hole; and
   17—second hole;
2—valve skirt; and
3—valve frame.

The accompanying drawings herein are incorporated into, and constitute a part of, this specification, illustrate embodiments consistent with the present disclosure, and serve to explain the principles of the present disclosure together with this specification.

### DESCRIPTION OF EMBODIMENTS

In order to better illustrate the purpose, technical solutions and advantages of the present disclosure, the present disclosure will be further described in detail below with reference to the drawings and embodiments. It should be understood that the specific embodiments described herein are used just to explain the present disclosure, but not to limit the present disclosure.

In the description of the present disclosure, unless otherwise expressly stipulated and defined, the terms such as “first” and “second” are merely used for descriptive purposes, but shall not be illustrated as indicating or implying relative importance. Unless otherwise specified or stated, the term “a plurality of” or “multiple” refers to two or more; the terms such as “connection”, “fixed/fixation”, etc. shall be illustrated in a broad sense. For example, “connection” may refer to “fixedly connection”, “detachably connection”, “integrally connection”, or “electrically connection”; and/or directly connection or indirectly connection through an intermediary. For one skilled in the art, the specific meanings of the above terms in the present disclosure can be understood according to specific circumstances.

In the description of this specification, it should be understood that the directional words such as “above/upper” and “below/lower” described in embodiments of the present disclosure are described from the perspective shown in the drawings, but cannot be illustrated as a limitation to the embodiments of the present disclosure. In addition, it should be understood in the context that an element is connected “above” or “below” another element, the element may be directly connected “above” or “below” the another element, or the element may be indirectly connected “above” or “below” the another element through an intermediate element.

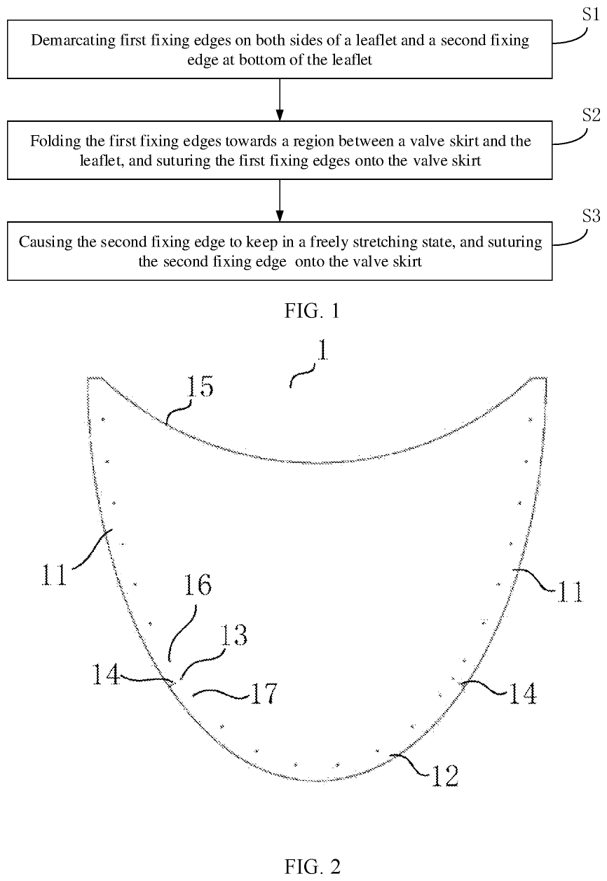

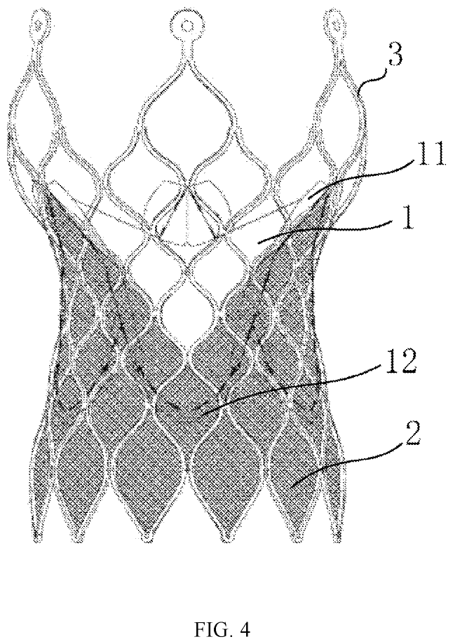

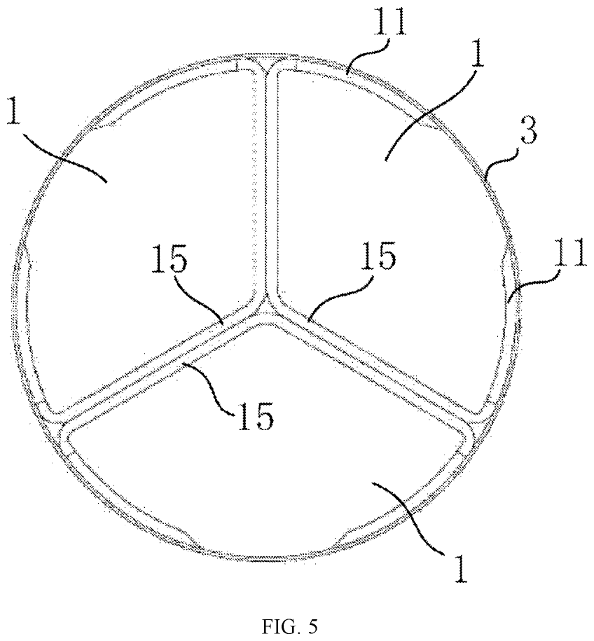

Referring to FIG. 1 to FIG. 7, embodiments of the present disclosure provide a method for suturing a valve leaflet. The method includes following steps.

Step S1, the first fixing edges 11 on both sides of the leaflet 1 and the second fixing edge 12 at the bottom of the leaflet 1 are demarcated.

As shown in FIG. 2, the leaflet 1 is in a fully unfolding state, the leaflet 1 can be sutured on the valve skirt 2 through the edges on both sides and the bottom of the leaflet 1, and the top edge of the leaflet 1 forms a free edge 15. The free edge 15, when impacted by the blood flow, can achieve two states: a closing state or an open state.

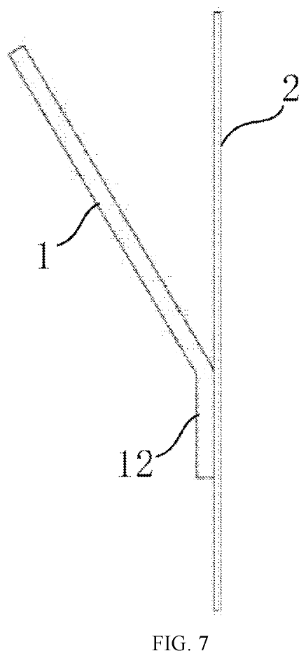

It can be understood that, as shown in FIG. 5, under a closing state of the valve, multiple leaflets 1 are spliced with one another to close the pathway of the valve. If the edges on both sides and the bottom of the leaflet 1 are fixed in the freely stretching state onto the valve skirt 2, as shown in FIG. 7, the free edges 15 of the each leaflet 1 will have a tendency to be separated and opened due to the tension of the leaflet 1, thereby resulting in delayed closure of the leaflet 1, increasing backward blood, and even resulting in accidents due to failure of forming a closing action.

When all edges of the leaflet 1 are folded towards the region between the leaflet 1 and the valve skirt 2, and are fixedly sutured with the valve skirt 2, the tension of the leaflet 1 can be used to accelerate the closure speed. However, the bottom edge of the leaflet 1 has no significant improving effect on the closure action of the leaflet 1. Moreover, after the bottom edge of the leaflet 1 is further folded between the leaflet 1 and the valve skirt 2, and is fixed with the valve skirt 2, the overall thickness of the leaflet 1 at bottom position thereof is increased, resulting in difficulty in the insertion of the valve into the delivery system.

To this end, in the present disclosure, the edges of the leaflet 1 is divided into two first fixing edges 11 on both sides of the leaflet 1 and the second fixing edge 12 at the bottom of the leaflet 1, so that only the first fixing edges 11 on both sides of the leaflet 1 can be folded without the second fixing edge 12 at the bottom of the leaflet 1, thereby reducing the thickness of the bottom of the leaflet 1 relative to both sides of the leaflet 1, and improving the insertion of the valve into the delivery system.

Step S2, the first fixing edges 11 is folded towards the region between the valve skirt 2 and the leaflet 1, and is sutured onto the valve skirt 2.

The region between the valve skirt 2 and the leaflet 1 refers to a region where the first fixing edges 11 is located when the first fixing edges 11 folded can bond with the valve skirt 2 after the leaflet 1 is sutured on the valve skirt 2.

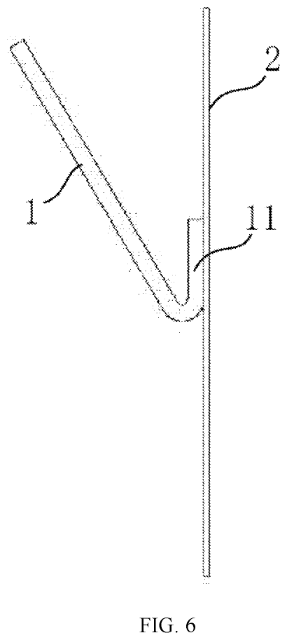

As shown in FIG. 6, the first fixing edge 11 is entirely located between the valve skirt 2 and the leaflet 1 after being folded, and is sutured with the valve skirt 2 through a suture line. Meanwhile, the leaflet 1 has a tendency to close towards the center of the valve through tension on both sides of the leaflet 1, thereby further achieving rapid closure through the action of blood flow.

Step S3, the second fixing edge 12 is caused to be a freely stretching state, and the second fixing edge 12 is sutured onto the valve skirt 2.

It can be understood that the second fixing edge 12 at the bottom of the leaflet 1 is not folded, but is sutured on the valve skirt 2 in a freely stretching state, as shown in FIG. 7. Meanwhile, the thickness of the leaflet 1 at the connection between the second fixing edge 12 and the valve skirt 2 is the thickness of the second fixing edge 12 itself. However, in the related art, since the entire edge of the leaflet 1 is folded towards the region between the leaflet 1 and the valve skirt 2, the thickness of the leaflet 1 at the bottom of the leaflet 1 is a sum of the thickness of the main body of the leaflet 1 and the thickness of the edge of the leaflet 1, as shown in FIG. 6, thereby increasing the volume of the valve at the bottom of the leaflet 1.

Therefore, compared with the related art, in this embodiment of the present disclosure, since the second fixing edge 12 at the bottom of the leaflet 1 is not folded, after the leaflet 1 is sutured on the valve skirt 2, the thickness at the bottom of the leaflet 1 is only the thickness of the second fixing edge 12, and does not overlap with the thickness of the main body of the leaflet 1, thereby effectively reducing the volume of the valve at the bottom of the leaflet 1.

In this embodiment, the continuous edge of the leaflet 1 is divided into the first fixing edge 11 and the second fixing edge 12, so that the first fixing edges 11 on both sides of the leaflet 1 can be folded towards the region between the valve skirt 2 and the leaflet 1, thereby accelerating the closure of the leaflet 1 through the tension effect of the leaflet 1. Meanwhile, the second fixing edge 12 at the bottom of the leaflet 1 is sutured in the free stretching state, so that the overall thickness of the bottom of the leaflet 1 after being fixed onto the valve skirt 2 can be reduced without affecting the valve closure effect, thereby improving the insertion of the valve into the delivery system and the implantation of the valve into the human body.

In an embodiment, the method includes the following steps.

Step S10, first fixing edges 11 is demarcated on both sides of a leaflet 1 and a second fixing edge 12 at the bottom of the leaflet 1.

Step S20, a transition hole position 13 is provided at a transition region formed between the first fixing edge 11 and the second fixing edge 12.

Step S30, the first fixing edges 11 are folded towards a region between the valve skirt 2 and the leaflet 1, and the first fixing edges 11 are sutured on the valve skirt 2.

Step S40, the second fixing edge 12 is caused to keep in a freely stretching state, and the second fixing edge 12 is sutured on the valve skirt 2.

Since the first fixing edge 11 is folded relative to the second fixing edge 12, the edge located at the border between the first fixing edge 11 and the second fixing edge 12 may be twisted, namely, the transition region is formed at a twisting position.

During suturing, after the suture line passes through the valve skirt 2 and the second fixing edge 12 in sequence, since the first fixing edge 11 is folded, the suture line cannot pass through the first fixing edge 11 in the direction of the valve skirt 2, or, after the suture line passes out of the folded position of the first fixing edge 11 in the direction towards the valve skirt 2, the suture line cannot continue to pass out of the valve skirt 2 due to being stopped by the first fixing edge 11.

Therefore, in this embodiment, the direction of the suture line can be changed by providing the transition hole position 13 in the transition region, so that the suture line can enter from the position between the folded first fixing edge 11 and the main body of the leaflet 1, and pass through the leaflet 1 and the leaflet skirt 2 in sequence to achieve suturing, thereby improving the route of the suture line and achieving the suturing effect on the leaflet 1.

In an embodiment, the method includes the following steps.

Step S100, first fixing edges 11 on both sides of the leaflet 1 and a second fixing edge 12 at the bottom of the leaflet 1 are demarcated.

Step S200, a transition hole position 13 is provided at the transition region formed between the first fixing edge 11 and the second fixing edge 12.

Step S300, an incision 14 is cut at the edge of the transition region.

Step S400, the first fixing edges 11 are folded towards the region between the valve skirt 2 and the leaflet 1, and the first fixing edges 11 is sutured on the valve skirt 2.

Step S500, the second fixing edge 12 is caused to keep in a freely stretching state, and the second fixing edge 12 is sutured onto the valve skirt 2.

It can be understood that, if the first fixing edge 11 and the second fixing edge 12 are a continuous edge of the leaflet 1 before the first fixing edge 11 is folded, portions of the first fixing edge 11 and the second fixing edge 12 in the transition region generate a torsional wrinkle when the first fixing edge 11 is folded, resulting in a risk of perivalvular leakage.

For this purpose, in this embodiment, by cutting one incision 14 in the transition region, the first fixing edge 11 can be conveniently folded to avoid the wrinkle and meanwhile, the problem of the perivalvular leakage is further avoided.

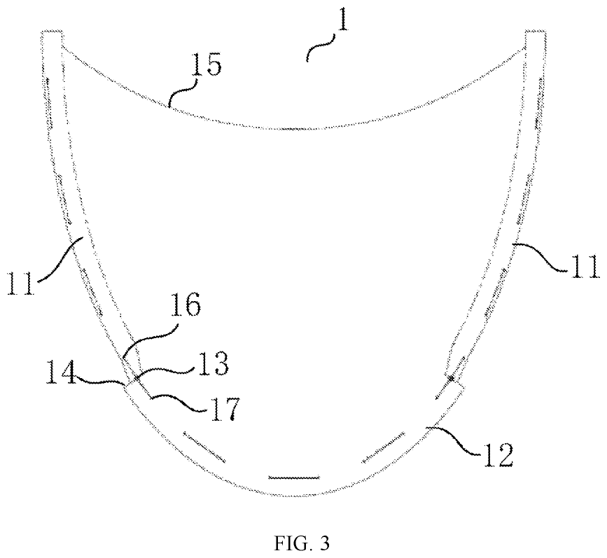

In an embodiment, a distance between one end of the incision 14 away from the edge of the transition region and the transition hole position 13 is ⅓ to ⅔ of a distance between the transition hole position 13 and the edge of the transition region, and the incision 14 extends from the edge of the transition region to a direction of the transition hole position 13, as shown in FIG. 2 and FIG. 3. In an embodiment, the distance between one end of the incision 14 away from the edge of the transition region and the transition hole position 13 is ½ of the distance between the transition hole position 13 and the edge of the transition region, and one end of the incision 14 away from the edge of the transition region is located in the center position between the transition hole position 13 and the edge of the transition region, so that the first fixing edge 11 has sufficient width to fold towards the region between the valve skirt 2 and the leaflet 1, thereby achieving the reliability and sealing of the connection between the first fixing edge 11 and the valve skirt 2. Meanwhile, communication between the incision 14 and the transition hole position 13 can be avoided, so that the suturing effectiveness of the suture line at the transition hole position 13 is ensured, thereby avoiding the perivalvular leakage due to the communication between the incision 14 and the transition hole position 13.

In an embodiment, as shown in FIG. 3, if the suture line passes through the valve skirt 2 and the second hole 17 on the second fixing edge 12 in sequence before entering the transition region, the method includes following steps.

Step A, after the suture line passes through the valve skirt 2 and the second hole 17 on the second fixing edge 12 in sequence, the suture line is caused to pass out of the transition hole position 13 in a direction towards the valve skirt 2.

Step B, the suture line is caused to pass through the first hole 16 on the first fixing edge 11 and the valve skirt 2 in sequence.

During suturing, when the suture line is routed from the second fixing edge 12 toward the first fixing edge 11, one needle of the suture line before entering the transition hole position 13 may sequentially pass through the valve skirt 2 and the second hole 17 on the second fixing edge 12 by the guide of a threading needle. The threading needle then passes out of the transition hole position 13 in the direction towards the valve skirt 2, and at this time, the threading needle is provided between the leaflet 1 and the valve skirt 2 without passing out of the valve skirt 2. The first fixing edge 11 is subsequently folded, so that the threading needle passes through the leaflet 1 from a position between the first hole 16 on the folded first fixing edge 11 and the main body of the leaflet 1, and further passes through the valve skirt 2, thereby achieving the suturing connection of the leaflet 1 at the transition region position. After the needle passes through the first hole 16 and the valve skirt 2 from a position between the folded first fixing edge 11 and the main body of the leaflet 1, the first fixing edge 11 may be bonded to the main body of the leaflet 1 by the tension thereof, thereby achieving the sealing effect.

In addition, since a gap is still formed between the incision 14 and the transition hole position 13, after the first fixing edge 11 is folded, a folded position of the first fixing edge 11 close to the incision 14 does not coincide with a routing path of the suture line on the transition hole position 13 and the first fixing edge 11. And after the suture line sequentially passes through the leaflet 1 and the leaflet skirt 2 from a position between the folded first fixing edge 11 and the main body of the leaflet 1, the suture line can be pressed on the first fixing edge 11 close to the incision 14, so that the first fixing edge 11 can be restrained from being bonded with the valve skirt 2.

Therefore, a natural conversion of routing on the second fixing edge 12 and the first fixing edge 11 is realized, the suturing quality of the leaflet 1 on the valve skirt 2 is ensured, and the perivalvular leakage is avoided.

In another embodiment, if the suture line passes through the second hole 17 on the second fixing edge 12 and the valve skirt 2 in sequence before entering the transition region, the method further includes following step.

After the suture line passes through the second hole 17 on the second fixing edge 12 and the valve skirt 2 in sequence, the suture line is caused to pass through the valve skirt 2 and the first hole 16 on the first fixing edges 11 in sequence without passing through the transition hole position 13.

The embodiments of the present disclosure further provides an interventional valve manufactured by the method for suturing the valve leaflet provided in above embodiments of the present disclosure. The interventional valve includes a valve frame 3, a valve skirt 2 and a leaflet 1. The valve skirt 2 covers the inner wall surface of the valve frame 3, and the first fixing edge 11 and the second fixing edge 12 of the leaflet 1 are fixedly sutured on the valve skirt 2.

By adopting the interventional valve manufactured by the method for suturing the valve leaflet provided by the embodiments of the present disclosure, the leaflet 1 can be smoothly opened when the blood flows forward, and the leaflet 1 can be quickly closed when the blood flows backward. Meanwhile, the thickness of the bottom of the leaflet can be effectively reduced after suturing, thereby reducing the local volume of the interventional valve at the bottom of the leaflet after compression, and improving the insertion of the interventional valve into a delivery system and the implantation of the interventional valve into the human body.

The above description merely describes some preferred embodiments of the present disclosure, and shall not be illustrated as to limit the present disclosure. For one skilled in the art, various changes and variations can be made to the present disclosure. Any modifications, equivalent replacements, improvements or the like made without departing from the principle and essence of the present disclosure shall fall within the protection scope of the present disclosure.

1. A method for suturing a valve leaflet, comprising:
demarcating first fixing edges (11) on both sides of a leaflet (1) and a second fixing edge (12) at bottom of the leaflet (1);
folding the first fixing edges (11) towards a region between a valve skirt (2) and the leaflet (1), and suturing the first fixing edges (11) onto the valve skirt (2); and
causing the second fixing edge (12) to keep in a freely stretching state, and suturing the second fixing edge (12) onto the valve skirt (2).

2. The method for suturing the valve leaflet according to claim 1, after demarcating the first fixing edges (11) on both sides of the leaflet (1) and the second fixing edge (12) at the bottom of the leaflet (1), further comprising:
providing a transition hole position (13) at a transition region that is formed between the first fixing edges (11) and the second fixing edge (12).

3. The method for suturing the valve leaflet according to claim 2, after providing the transition hole position (13), further comprising:
cutting an incision (14) at an edge of the transition region.

4. The method for suturing the valve leaflet according to claim 3, wherein a distance between one end of the incision away from the edge of the transition region and the transition hole position is ⅓ to ⅔ of a distance between the transition hole position and the edge of the transition region, and the incision (14) extends in a direction from the edge of the transition region to the transition hole position (13).

5. The method for suturing the valve leaflet according to claim 2, when a suture line passes through the valve skirt (2) and the second fixing edge (12) in sequence before entering the transition region, further comprising:
causing the suture line to pass through the transition hole position (13) in a direction towards the valve skirt (2) after the suture line passes through the valve skirt (2) and the second fixing edge (12) in sequence; and
causing the suture line to pass through the first fixing edges (11) and the valve skirt (2) in sequence.

6. The method for suturing the valve leaflet according to claim 2, when the suture line passes through the second fixing edge (12) and the valve skirt (2) in sequence before entering the transition region, further comprising:
causing the suture line to pass through the valve skirt (2) and the first fixing edges (11) in sequence without passing through the transition hole position (13), after the suture line passes through the second fixing edge (12) and the valve skirt (2) in sequence.

7. An interventional valve manufactured by a method for suturing a valve leaflet,
wherein the method comprises:
demarcating first fixing edges (11) on both sides of a leaflet (1) and a second fixing edge (12) at bottom of the leaflet (1);
folding the first fixing edges (11) towards a region between a valve skirt (2) and the leaflet (1), and suturing the first fixing edges (11) onto the valve skirt (2); and
causing the second fixing edge (12) to keep in a freely stretching state, and suturing the second fixing edge (12) onto the valve skirt (2);
the interventional valve comprises: a valve frame (3), the valve skirt (2) and the leaflet (1), wherein the valve skirt (2) covers an inner wall surface of the valve frame (3), and first fixing edges (11) and a second fixing edge (12) of the leaflet (1) are fixedly sutured on the valve skirt (2).
